(19) United States
(12) Patent Application Publication
(10) Pub. No.: US 2007/0053839 A1
(43) Pub. Date: Mar. 8, 2007

(54) Directing cells to target tissues organs

(76) Inventor: Jianyi Zhang, Plymouth, MN (US)

Correspondence Address: FISH & RICHARDSON P.C., PO BOX 1022, MINNEAPOLIS, MN, 55440-1022, US

(21) Appl. No.: 10/560,274

(22) PCT Filed: Jun. 10, 2004
(86) PCT No.: PCT/US04/18776

§ 371 Date: Oct. 31, 2006

(30) Foreign Application Priority Data
Jun. 12, 2003 (US) 60478568
Jun. 18, 2003 (US) 60479384

(51) Int. Cl. A61K 49/00; A61B 5/05

(52) U.S. Cl. 424/9.1; 600/420

(57) ABSTRACT

The invention provides for methods of directing cells to a damaged tissue or organ in an individual, and further provides for methods of monitoring such cells in the individual. The invention also provides compositions for tagging cells such that the cells can be directed to the damaged tissue or organ. In addition, the invention provides for isolated stem cells that have been tagged such that the tagged cells can be directed to a damaged tissue or organ.

### TECHNICAL FIELD

This invention relates generally to directing cells, and more specifically to directing cells to injured or diseased tissues or organs.

### BACKGROUND

Heart failure is an increasingly common clinical problem that affects 8 of every 100 individuals past the age of 70 years. Mechanical overload resulting from regional loss of functioning myocardium secondary to infarct can result in asymptomatic left ventricular dysfunction of long duration. During this time, myocyte hypertrophy is commonly seen, but contractile function of isolated myocytes may remain normal despite abnormal chamber function. However, prolonged overload often leads to the development of overt congestive heart failure and the appearance of contractile dysfunction of isolated myocytes. In a general sense, the molecular and cellular basis for the syndrome of progressive heart failure results from the inability of damaged and apoptotic myocytes to be replaced, since cardiac myocytes are generally thought to be terminally differentiated.

### SUMMARY

The invention establishes a system for directing and non-invasive tracking of transplanted stem cells in vivo. Stem cells can be tagged and labeled to direct the stem cells to the target tissue or organ and to monitor their location, respectively. Methods of the invention can be used for cellular therapy in regenerative medicine and specifically can be used to treat transmural myocardial infarct as well as cardiac failure secondary to postinfarction LV remodeling.

In one aspect, the invention provides a method of directing cells to a damaged or diseased tissue or organ in an individual. Such a method includes providing a tagged cell, wherein the cells are tagged with a target cell binding member; and introducing the tagged cell into the vasculature of the individual. Such a method directs the cells to the damaged or diseased tissue or organ.

The cells used in the methods of the invention can be autologous, allogeneic, or xenogeneic relative to said individual. For example, the cells used in the methods of the invention can be stem cells. Representative stem cells include mesenchymal stem cells (MSCs), and endothelial progenitor stem cells (EPCs). Cells generally are introduced into an individual via a coronary vein, a peripheral vein, or a coronary artery of the individual.

Representataive target cell binding members include annexin, an antibody having specific binding affinity for cardiac-specific troponin T, an antibody having specific binding affinity for cardiac-specific troponin I, an antibody having specific binding affinity for skeletal muscle-specific troponin T, an antibody having specific binding affinity for skeletal muscle-specific troponin I, and an antibody having specific binding affinity for myosin.

Examples of damaged tissues or organs include mycocardial tissue, pericardial tissue, pancreatic tissue, kidney tissue, skeletal muscle tissue, central nervous system tissue, and liver tissue.

In an embodiment of the invention, tagged cells also can include an imaging agent. Representative imaging agents include monocristalline iron oxide nanoparticle (MION), superparamagnetic iron oxide particles (SPIO), and ultra small superparamagnetic iron oxide (USPIO). Such an imaging agent can be used for imaging the tagged cells.

In another aspect, the invention provides a method of delivering stem cells to a myocardial infarction in an individual. Such a method includes providing tagged stem cells, wherein the stem cells are tagged with annexin; and introducing the tagged stem cell into the vasculature of the individual. Such a method thereby delivers the stem cells to the myocardial infarction. Representative stem cells include MSCs and EPCs.

In yet another aspect, the invention provides a composition that includes at least one linker moiety; and at least one target cell binding member. Representative target cell binding members include annexin, an antibody having specific binding affinity for cardiac-specific troponin T, an antibody having specific binding affinity for cardiac-specific troponin I, an antibody having specific binding affinity for skeletal muscle-specific troponin T, an antibody having specific binding affinity for skeletal muscle-specific troponin I, and an antibody having specific binding affinity for myosin. A composition of the invention can further include an imaging agent such as MION, SPIO, and USPIO.

A composition of the invention can include instructions for tagging cells with the target cell binding member using the linker, wherein the cells are stem cells harvested from an individual, and further can include instructions for performing an autologous transplant on the individual with the cells after the tagging.

In still another aspect, the invention provides isolated stem cells, wherein the stem cells are tagged with a heterologous target cell binding member. Such stem cells can be further labeled with an imaging agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows histograms of flow cytometry of mesenchymal stem cells (MSCs) with and without tagging (bottom row). Panel A demonstrates that MSCs without tags interacted with FITC-anti-annexin antibody only. The fluorescence counts represent the FITC-IgG. Panel B demonstrates that MSCs tagged with anti-CD44 antibody crosslinked to annexin interacted with FITC-IgG. The fluorescence counts represent the FITC-IgG. Panel C demonstrates that MSCs tagged with anti-CD44 antibody crosslinked to annexin interacted with FITC-anti-annexin antibody. The top row shows the histograms from Panel A, B, and/or C combined as indicated.

Like reference symbols in the various drawings indicate like elements.

### DETAILED DESCRIPTION

The invention establishes a system for directing and non-invasive tracking of transplanted stem cells in vivo. For example, autologous stem cells can be tagged with annexin and labeled with an imaging agent, which can direct the stem cells to the target organ and allow for non-invasive monitoring of the stem cells (e.g., using magnetic resonance imaging (MRI)), respectively. Such tagged and labeled stem cells can be used clinically to increase engraftment of the transplanted stem cells, and to allow for non-surgical transplantation. Methods of the invention can be used to treat damaged (injured) or diseased tissues or organs such as, but not limited to heart, liver, kidney, muscle, or pancreas using cellular therapy such as stem cells. For example, methods of the invention can be used to treat transmural myocardial infarct as well as cardiac failure secondary to postinfarction left ventricular (LV) remodeling.

Stem Cells

Stem cells are defined as cells that have extensive, sometimes indefinite, proliferation potential, that can differentiate into several cell lineages, and that can re-populate tissues upon transplantation. The quintessential stem cell is the embryonal stem (ES) cell, as ES cells typically have unlimited self-renewal and multipotent differentiation potential. ES cells are derived from the inner cell mass of a blastocyst, or can be derived from primordial germ cells from a post-implantation embryo (embryonal germ (EG) cells). ES and EG cells have been derived from mice, non-human primates, and humans. When introduced into mouse blastocysts or blastocysts from other animals, ES cells can contribute to all tissues of the mouse. When transplanted into post-natal animals, ES and EG cells generate teratomas, which again demonstrates their multipotency. ES and EG cells can be identified by positive staining with anti-SSEA-1 and anti-SSEA-4 antibodies (Thomson et al., 1998, Science, 282:114). At the molecular level, ES and EG cells express a number of transcription factors highly specific for these undifferentiated cells including oct-4 and Rex-1. Another hallmark of ES cells is the presence of telomerase, which provides these cells with unlimited self-renewal potential in vitro.

Stem cells have also been identified in many tissues. The best characterized is the hematopoietic stem cell, while neural, gastrointestinal, epidermal, hepatic and mesenchymal stem cells (MSCs) also have been described. Endothelial progenitor stem cells (EPCs) also have been described. Compared with ES cells, tissue specific stem cells have less self-renewal ability and, although they can differentiate into multiple lineages, they are usually not multipotent.

Until recently, it was thought that tissue specific stem cells could differentiate into cells of only that type of tissue. However, a number of recent reports have suggested that adult organ-specific stem cells may be capable of differentiating into cells of different tissues. Two studies have shown that cells infused at the time of a bone marrow transplantation can differentiate into skeletal muscle (Ferrari et al., 1998, Science, 279:528-30; Gussoni et al., 1999, Nature, 401:390-4). Other studies suggest that stem cells from one embryonal layer (for instance splanchnic mesoderm) can differentiate into tissues from a different embryonal layer. For instance, endothelial cells or their precursors that are detected in humans or animals that underwent marrow transplantation are at least in part derived from the marrow donor (Takahashi et al., 1999, Nat. Med., 5:434-8; Lin et al., 2000, J. Clin. Invest., 105:71-7). Even more surprising are reports demonstrating that hepatic epithelial cells and biliary duct epithelial cells in both rodents and humans are derived from the donor marrow (Wang et al., 2003, Nature, 422:897-901 and references therein). Likewise, neural stem cells can differentiate into hematopoietic cells (Orlic et al., 2001, Nature, 410:701-5; Jackson et al., 2001, J. Clin. Invest., 107:1395-1402). Finally, it has been reported that neural stem cells injected into blastocysts can contribute to all tissues of a chimeric mouse (Asahara et al., 1999, Circ. Res., 85:221-8).

Most studies that show differentiation of stem cells into cell types outside the normal differentiation process have shown that this occurs almost exclusively in organs that have been damaged: ischemia for endothelial engraftment (Takahashi et al., 1999, Nat. Med., 5:434-8), cirrhosis for liver and bile duct engraftment (Wang et al., 2003, Nature, 422:897-901), toxin administration (Ferrari et al., 1998, Science, 279:528-30), or muscular dystrophy (Gussoni et al., 1999, Nature, 401:390-4) for muscle engraftment, or when the organ is growing.

Examples of stem cells include mesenchymal stem cells (MSCs) and endothelial progenitor stem cells (EPCs), as well as numerous others available commercially or from public depositories (e.g., American Type Culture Collection, Manassas, Va.). See also U.S. Pat. Nos. 5,843,780 and 6,200,806. Although stem cells would likely be used in most clinical settings, non-stem cells also can be tagged as described herein and used in the methods of the invention.

Tagging Stem Cells

The methods of the invention allow for targeted delivery of stem cells to a damaged or diseased tissue or organ. Targeted delivery of stem cells is accomplished by tagging the stem cells with a “target cell binding member.” As used herein, “target cell binding member” refers to a polypeptide (e.g., an antibody) or other macromolecule (e.g., a carbohydrate) that has binding affinity for a second binding member (e.g., polypeptide) that is available for binding in target cells of the damaged or diseased tissue or organ. Such second binding members are generally not available for binding in cells of tissues or organs that are not damaged or diseased. A heterologous target cell binding member is a binding member that is not found attached to the stem cells in nature. Cells of damaged or diseased tissues or organs include those cells undergoing death. A cell can undergo death due to injury or suicide (i.e., apoptosis).

One example of a target cell binding member that can be used to tag stem cells is annexin V. Annexin V binds to exteriorized phosphatidylserine (PS) with a very high affinity (Kₐ=7 nM). This tight binding has been used to identify apoptotic cells characterized by PS exteriorization. Annexin V also binds to necrotic cells. Although coagulation necrosis is characteristic of myocardial infarction, large numbers of apoptotic myocytes are found admixed with necrotic cells in the infarct center, particularly during reperfusion. Therefore, annexin V deposition can identify a region of acute myocardial infarction. Radiolabeled annexin V also has been used for non-invasive detection of cardiac allograft rejection.

Antibodies also can be used as target cell binding members. Antibodies have been used to deliver isotopes in radiation medicine, and to direct cytotoxic drug compounds to specific host tissue cells or tumor cells in oncology. Therefore, antibodies having specific binding affinity for a protein that becomes available for binding upon cell death can be used in the present invention. Representative proteins that become available for binding upon injury or disease of one or more cell include, but are not limited to, cardiac-specific troponin T, cardiac-specific troponin I, skeletal muscle-specific troponin T, skeletal muscle-specific troponin I, and myosin.

There are many examples of proteins that can be used as target cell binding members or that can be used to generate target cell binding members. For example, the pathological changes in different phases of post-infarction myocardium are orchestrated by necrosis, apoptosis, and other inflammatory responses including the cytokine cascade, growth factors, chemoattractants, adhesion molecules, cell infiltration, angiogenesis, and the release of cellular components, e.g., myosin, or troponin T. Therefore, it is possible to direct stem cells to a damaged tissue or organ (e.g., an infarcted myocardial area) using a target cell binding member that binds to a second binding member in or on cells of the target tissue or organ.

“Tagging” as used herein refers to the act of attaching a target cell binding member to a stem cell. Stem cells can be tagged with a target cell binding member using a number of different “linkers.” For example, an antibody having specific binding affinity for a cell-surface protein can be used. For example, anti-CD44 antibodies can be attached to a target cell binding member and used to link the binding member to a mesenchymal stem cell. Alternatively, anti-CD31 antibodies or anti-CD34 antibodies can be attached to a target cell binding member and used to link the binding member to circulating EPCs. In addition, to increase the number of sites available to attach target cell binding members and/or imaging agents, the antibody can be biotinylated (before or after the antibody is attached to the stem cell), and contacted with avidin-target cell binding member complexes. Avidin has multiple binding sites, and therefore can accommodate multiple moieties (e.g., multiple target cell binding members, and/or one or more imaging agents).

The ability of a target cell binding member to target a damaged tissue or organ in an individual can be evaluated using the in vitro methods and animal models described herein.

Methods of Delivering Tagged Stem Cells

Once stem cells are tagged, they can be delivered to the vasculature of an individual using several different routes. Stem cells can be introduced into an individual through an anterior intraventricular vein catheter. It can be advantageous to close the coronary vein by ligature after introducing the stem cells. Alternatively, stem cells can be introduced through the coronary artery. Generally, 100 to 50 million stem cells are transplanted into an individual (e.g., 1000 cells, 10,000 cells, 100,000 cells, 1,000,000 cells, 10,000,000 cells, or 50,000,000 cells). Methods for introducing a catheter into the vasculature of an individual are known to those of skill in the art.

The stem cells delivered to an individual can be from a variety of sources. Relative to the individual receiving the stem cells, the stem cells can be allogeneic (i.e., from the same species (e.g., human) but a different individual (e.g., a close relative)) or xenogeneic (i.e., from a different species (e.g., a swine or non-human primate) than that of the recipient individual (e.g., a human)). In the most common clinical application, the stem cells would be autologous. For example, stem cells can be obtained from an individual (e.g., at the time of treatment or collected at birth), tagged, and labeled if so desired, and introduced back into the same individual.

Methods of Non-Invasive Monitoring of Stem Cells

Various types of MRI methods can be used in conjunction with an appropriate imaging agent to monitor the stem cells once they have been introduced into an individual. Imaging agents include a physiologically compatible metal chelate compound consisting of one or more cyclic or acyclic organic chelating agents complexed to one or more metal ions, iodinated organic molecules, chelates of heavy metal ions, gas-filled bubbles, radioactive molecules, organic and inorganic dyes, and metal-ligand complexes of paramagnetic forms of metal ions. Chelating agents for MRI are known in the art, and include magnevist gadopentetate dimeglumine (DTPA), dotarem gadoterate meglumine (DOTA), omniscan gadodiamide (DTPA-BMA), and ProHance gadoteridol (HP-DO3A). Specific examples of imaging agents include monocristalline iron oxide nanoparticle (MION), superparamagnetic iron oxide particles (SPIO), and ultra small superparamagnetic iron oxide (USPIO). Imaging agents are available commercially from, for example, Advanced Magnetics (Cambridge, Mass.). Methods for introducing imaging agents into cells are well known in the art.

In MRI, the image of an organ or tissue is obtained by placing a subject in a strong external magnetic field and observing the effect of this field on the magnetic properties of the protons (hydrogen nuclei) contained in and surrounding the organ or tissue. The proton relaxation times, termed T₁ and T₂, are of primary importance. T₁ (also called the spin-lattice or longitudinal relaxation time) and T₂ (also called the spin-spin or transverse relaxation time) depend on the chemical and physical environment of organ or tissue protons and are measured using the Rf pulsing technique; this information is analyzed as a function of distance by computer which then uses it to generate an image.

In order for an imaging agent to effectively image, the agent must be capable of enhancing the relaxation rates 1/T₁ (longitudinal, or spin-lattice) and/or 1/T₂ (transverse, or spin-spin) of water protons or other imaging or spectroscopic nuclei, including protons, on other biomolecules. Relaxivities R₁ and R₂ are defined as the ability to increase 1/T₁ or 1/T₂, respectively, per mM of metal ion (mM⁻¹s⁻¹). The most common form of clinical MRI is water proton MRI. In addition to increasing the 1/T₁ or 1/T₂ of tissue nuclei via dipole-dipole interactions, imaging agents can affect two other magnetic properties and thus can be of use clinically. First, an iron particle or metal chelate of high magnetic susceptibility, particularly chelates of Dy, Gd, or Ho, can alter the MRI signal intensity of tissue by creating microscopic magnetic susceptibility gradients. Second, an iron particle or metal chelate can also be used to shift the resonance frequency of water proton or other imaging or spectroscopic nuclei, including protons, on other biomolecules. Depending upon the strategy used, zero to three open coordination sites can be employed.

For descriptions and reviews of imaging agents, the introduction of imaging agents into cells, and imaging techniques, see, for example, Lauffer, 1987, Chem. Rev., 87:901-27; Caravan et al., 1999, Chem. Rev., 99:2293-2352; and U.S. Pat. No. 4,951,675.

Compositions and Articles of Manufacture

The invention also includes compositions for tagging stem cells. A composition of the invention can include at least one linker moiety; and at least one target cell binding member. Representative target cell binding members are described above, and include annexin, an antibody having specific binding affinity for cardiac-specific troponin T, an antibody having specific binding affinity for cardiac-specific troponin I, an antibody having specific binding affinity for skeletal muscle-specific troponin T, an antibody having specific binding affinity for skeletal muscle-specific troponin I, and an antibody having specific binding affinity for myosin. Similarly, linkers are described above, and include antibodies having specific binding affinity for a cell-specific surface antigen, and avidin/biotin pairs. A composition of the invention also can include an imaging agent such as those described above for monitoring the stem cells in vivo. Specific examples of imaging agents include MION, SPIO, and USPIO.

An article of manufacture of the invention generally includes compositions as described above and packaging material (e.g., vials, or containers). Articles of manufacture can further include written instructions. The instructions can describe how to tag cells with the linker and the target cell binding member. The instructions can be specific to tagging cells harvested from an individual, and can additionally include instructions for performing an autologous transplant on the individual with the tagged cells.

Articles of manufacture of the invention also can include additional reagents for tagging and/or labeling stem cells. Additional reagents can be buffers, enzymes, co-factors, or materials to confirm the tagging and/or labeling. Articles of manufacture of the invention also can include materials or reagents for harvesting stem cells from an individual and preparing them for the tagging and/or labeling process. Further, articles of manufacture of the invention can include materials for monitoring the stem cells in the individual (e.g., additional contrast agents).

The invention will be further described in the following examples, which do not limit the scope of the invention described in the claims.

### EXAMPLES

### Example 1

### Experimental Groups

Animals in Group 1 (n=10 pigs) were exposed only to coronary ligation with no cell transplantation. Animals in Group 2 (n=10 pigs) were exposed to postinfarction LV remodeling and were transplated with autologous MSCs. Animals in Group 3 (n=15 pigs) received MSCs tagged with annexin. Animals in Group 4 (n=15 pigs) received autologous MSCs tagged with annexin and labeled with MION.

Briefly, Yorkshire swines (45 days of age; ˜10 kg) were anesthetized with intravenous sodium pentobarbital (20 mg/kg, intravenously (iv)). A left thoracotomy was performed. Approximately 0.5 cm of the left anterior descending (LAD) coronary artery distal to the first diagonal vessel was dissected free and a silicone elastomer catheter (0.3 mm id) is placed into the LAD coronary artery. For the animals in Group 1, the chest was closed in layers and the animals were allowed to recover. For the animals in Groups 2, 3, and 4, the LAD coronary artery was occluded by either a ligature proximal to the catheter or a ligature at the origin of the anterior intra ventricular vein from the coronary sinus, and 10 million MSCs (autologous cells in 0.5 ml saline solution) were slowly injected into the LAD coronary artery through the catheter. The catheter was then removed and the artery repaired. Following 2 hours of LAD coronary artery occlusion, the occlusion ligature was removed. This allowed 2 hours of dwelling time for the MSCs being exposed to the ischemic myocardium. Reperfusion arrhythmias were treated with defibrillation. The chest was then closed in layers. Animals that received transplanted autologous MSCs received immunosupression with Cyclosporine A (15 mg/kg daily with food). All animals were examined using MRI or magnetic resonance spectroscopy (MRS) once every two weeks, and underwent a final study 8 weeks after myocardial infarction.

### Example 2

### Methods of Monitoring the Labeled Stem Cells and Their Effects on the Heart

For the non-invasive studies, the animals were anesthetized with sodium pentobarbital (30 mg/kg, iv) following sedation with ketamine (20 mg/kg, intramuscularly (im)). At the final MRI study, a catheter was placed into the left femoral artery and advanced into the LV chamber for LV pressure recording. Following the MRI study, the femoral catheter was removed and the wound repaired.

Non-Invasive ³¹P-MRS

A technique was been developed using ³¹P-MRS study with an external coil in a closed chest dog model to examine myocardial phosphates non-invasively. In this non-invasive study, the transmural distribution of ³¹P metabolites from cylindrical regions across the IV wall of a closed-chest canine model were measured. MRI studies were conducted on a 4.7 T/40 cm SISCO system. When spectroscopic imaging was implemented with the Fourier Transform approach, spectra originated from rectangularly shaped regions with potentially significant errors from cross-voxel contamination. In the present experiments, Fourier Series Window (FSW) and selective Fourier transform methods weighted the data sampling with a desired filter, thereby eliminating the cross-voxel contamination due to the Fourier transform point-spread function; spectra were generated from spatially localized voxels of predetermined shape, the position of which can be shifted arbitrarily in the phase-encode directions. In this study, the 3-D B₀ FSW technique was used to define cylindrical voxels; this voxel shape not only conforms well to the geometry of the IV wall, but also requires fewer phase-encode steps than required for a rectangular voxel. A 7.3 cm diameter surface coil was utilized for ³¹P spectroscopy. Anatomical images were acquired with a dual-loop ¹H coil utilizing a fast gradient-echo sequence, with a magnetization transfer preparation period generating high contrast between tissue and blood.

Nine adult mongrel dogs weighing 13-26 kg were anesthetized and intubated. A catheter was introduced into the femoral vein and advanced to monitor LV pressure. The animals were placed in the prone position on the coil platform, with the heart directly over the ³¹P surface coil. To more clearly demonstrate the distinction between skeletal and heart muscle, the skeletal muscle of the chest wall was made ischemic by applying pressure to the ribs as the animal was positioned securely on the platform. The 3-D B₀ FSW sequence for ³¹P spectroscopy was performed over a 10×10×6 cm³ FOV with 5-term circulate coefficients to obtain cylinder diameters with full width of half-maximum signal intensity (FWHM of 19 mm, and 9-term rectangular coefficients to obtain cylinder heights of 5 mm (cylinder FWHM volume=1418 mm³). Data acquisition was synchronized to the cardiac cycle only, as respiratory motion was found to be minimal in the region of the LV wall studied. The radiofrequency (RF) pulse length was 33 μs, with 1 ms phase-encode gradients incremented by 0.091 G/cm to define the cylinder diameter, and by 0.152 G/cm to define the cylinder height, for a total of 681 distinct gradient combinations. A total of 1959 transients were collected within 26 min. The number of data acquisitions for each phase-encoded step was weighted according to the Fourier coefficients; differences between the actual coefficients and the integer number of accumulations were accounted for by multiplying the resultant signals with correction coefficients. A spectrum from a single voxel was generated by summation with respect to the phase-encode domain; spectra from arbitrarily defined spatial locations were generated by voxel-shifting the data with post-acquisition processing.

Nuclear Magnetic Resonance (NMR) Spectroscopy

Spatially localized ³¹P NMR spectroscopy was performed in open chest animals using the RAPP-ISIS method (see, for example, Wang et al., 2002, Amer. J. Pathol., 161:565-74 and references therein). Creatine phosphate (CP), ATP, and Pi levels corresponding to the integrals of each resonance peak were serially monitored throughout the study. The chemical shift of Pi relative to that of CP was used to calculate cytosolic pH. Mg²⁺ was determined from the chemical shift between α- and β-ATP (Verhoven et al., 1995, J. Exp. Med., 182:1597-1601). An high pressure liquid chromatography (HPLC)-measured ATP value obtained from an epicardial biopsy at the end of the experiment, taken together with the integrals of the peaks on the immediate pre-biopsy sub-epicardial spectrum peak integrals, was used to quantify all spectra.

Calculating Myocardial Free ADP Levels

The myocardial free ADP level was calculated from the creatine kinase equilibrium expression using an equilibrium constant of 1.66×10⁹, and cytosolic pH=7.1: [ADP]=([ATP][CR_{free}])/([CP][H⁺K_{eq}) (Verhoven et al., 1995, J. Exp. Med., 182:1597-1601). CP and ATP values were obtained from spectra calibrated by the biopsy measured ATP levels. Free creatine was calculated by subtracting the CP values from the biopsy obtained measurement of total creatine.

H-MRS Measurements

¹H-MRS deoxymyoglobin measurements were performed as described above in open chest animals using the double tuned surface coil placed on the epicardial surface in the LAD perfusion bed. Because of the short T₁ and T₂ of this Mb-δ signal, spatial localization could not be performed with phase encoding and other strategies that required gradient switching following signal excitation. Therefore, transmural localization was performed using 1D frequency encoding perpendicular to the LV wall surface underneath the surface coil and letting the small coil dimensions restrict the signal in the other two dimensions on the plane of the coil. The frequency encoding was performed by turning on the gradient prior to the first signal excitation and leaving it on during the entire acquisition and all subsequent signal excitations and data acquisitions during signal averaging. This strategy took advantage of the large frequency shift between the water and Mb-δ resonance. The gradient magnitude was ˜0.1 to 0.2 G/cm so that across the typically 1 cm thick LV wall, the frequency difference was ˜450 to 900 Hz. Myoglobin saturation (%) is defined as 100 (measured deoxymyoglobin resonance intensity/deoxymyoglobin resonance intensity during total occlusion) and is converted to P_{O2} using the myoglobin saturation-P_{O2} curves as previously reported (Zhang et al., 2001, Am. J. Physiol. Heart Circ. Physiol., 280:H318-H326).

MRI Cine Technique

The parameters of the segmented cine sequence at 1.5T were: TR/TE/flip angle=33 ms/6.1 ms/25 degrees with a FOV=17.5 cm and a matrix of 87×128 (pixel size: 2 mm×1.4 mm) interpolated to 256×256 and slice thickness of 7-10 mm. This 10-minute protocol provided high signal to noise movie-like cine sequences covering the entire heart.

In order to obtain high-resolution anatomical heart images, multi-slice spin echo images were acquired to cover the entire heart. These images permit the precise delineation of the extent of the scar region of the heart.

The imaging data were evaluated using an automatic segmentation program. Ventricular volumes, ejection-fraction, LV diastolic and systolic volumes were obtained. Absolute myocardial mass from multi-slice, multi-phase MR cine images were then automatically calculated. The left ventricular end-diastolic volume (V_{d}) and end-systolic volume (Vₛ) of each slice was represented by the area enclosed by the endocardium. The total left ventricular volume was computed by adding the volumes of all slices. LV EF was calculated by 100% X (V_{d}−Vₛ)/V_{d}. Inter observer and intra observer error for the calculations of LV mass and LV volumes have been previously shown to be less than 3 gm and 3 ml, respectively. Meridianal wall stress was computed from the LV pressure and simultaneously obtained LV radius measurements from short axis view of LV MRI (LV cavity diameter and average thickness the remote LV wall) as previously described (Grossman et al., 1975, J. Clin. Invest., 56:56-84).

Gd-EDTA enhanced MRI has been demonstrated as a reliable method to evaluate the myocardial viability (Kim et al., 1999, Circulation, 100:1992-2002). At an initial MRI study, infarct size can be quantitated by injecting (via the left atrial line) Gd-MP (an MRI contrast agent which has been used to examine myocardial viability) at ˜3 hours post-infarction. This technique correlated well with triphenyltetrazolium chloride (TTC) staining results; validation studies comparing Gd-MP estimation of infarct size with TTC-measured infarct. The ratio of mass of myocardium demonstrating Gd-MP brightness to total mass of LV myocardium was considered to be the % LV infarcted. The severity of the initial myocardial damage indicated by this valuable was then analyzed with the valuables, which reflected the severity of LV remodeling, ejection fraction, as well as myocardial bioenergetics in each group. Finally, this ratio was compared with the final scar weight.

All MRI studies were performed on a standard Siemens Medical System VISION® operating at 1.5 Tesla. All of the imaging sequences were gated with regard to the electrocardiographic signal obtained from leads placed on the shaved skin surface while respiratory gating was achieved by triggering the ventilator to the cardiac cycle between data acquisitions.

### Example 3

### In vitro Protocol

In vitro experiments were carried out to ensure that the respective cell labeling technique (β-glactosidase or MION) does not alter the characteristics of the MSCs. The MSCs were labeled with MION as described previously. Alternatively, the MSCs were tagged with nanoparticles on the cell surface using an annexin/MION complex.

The following methods were used to tag the stem cells with annexin V. Anti-CD44 antibodies were bound to MSCs by adding 2.5 μg of mouse anti-pig-CD44 IgG₂ₐ (the 1^{st} antibody) (VMRD, Inc.; Catalog No. PORC24A) to 5×10⁵ MSCs in 100 μl of 1% bovine serum albumin/phosphate buffer saline (pH 7.2-7.4) (BSA/PBS) in a 5 ml tube, mixing well, incubating at 4° C. for 30-40 min, washing with 5 ml of BSA/PBS to remove free antibody, and centrifuging at 1200 rpm for 5 min. The pellet of MSCs bound anti-pit-CD44 mAb was suspended in 100 μl of BSA/PBS.

A conjugate of biotinylated rabbit anti-mouse IgG (the 2^{nd} antibody) and streptavidin was prepared as follows. In parallel, 1.25 μg of biotinylated rabbit anti-mouse IgG (Catalog No. EO464, DAKO) and 100 μg of streptavidin (Catalog No. 62300, ICN) were combined in a 1.5 ml eppendorf tube containing up to 500 μl of BSA/PBS, mixed well, and incubated at room temperature for 60 min in the dark.

A heteroaggregate was formed by adding the conjugate (streptavidin x biotinylated antibody) obtained as described above to the 1^{st} antibody-bound MSCs, mixing well and incubating at 4° C. for 30 min in the dark. The cells were then washed with 5 ml of BSA/PBS to remove any unbound materials.

Annexin V was bound to the heteroaggregate-linked cells using streptavidin as a bridge. First, 0.6 μg of annexin V was added to conjugated biotin (Catalog No. PF036, Oncogene). The annexin-biotin complex was then added to the heteroaggregate-linked MSCs, and mixed and incubated at 4° C. for 30 min in the dark. BSA/PBS was used to wash and collect the pellet after centrifugation.

To test the binding efficiency of the dual specificity of the heteroaggregated antibody (anti-CD44 x annexin V), 1.5 μg of goat anti-annexin V IgG (Catalog No. Annexin V (C-20) SC-1928, Santa Cruz Biotechnology, Inc.) and 1 μl of rabbit anti-goat IgG-FITC (Product No. F 7363, Sigma) were combined, and incubated at 4° C. for 30 min. The mixture was washed with BSA/PBS, centrifuged, and the pellet suspended in 0.1 ml BSA/PBS containing 0.4 ml of fixative solution. The mixture was then analyzed by fluorescence-activated cell sorting (FACS). Goat IgG, instead of goat anti-annexin V, was used as a negative control.

The following describes the procedure for labeling stem cells with ultra small superparamagnetic iron oxide (USPIO) particles for imaging. Briefly, fridex (5 mg/ml) was co-incubated with fugene (1 μl/ml) for 30 minutes in serum-free modified Dulbecco/Vogt modified Eagle's minimal essential medium (DMEM) consisting of 60% low-glucose DMEM (Gibco BRL), 40% MCDB-201 (Sigma), 1× insulin transferin selenium, 1× linoleic acid-bovine serum albumin (LA-BSA), 0.05 μM dexamethasone (Sigma), 0.1 mM ascorbic acid 2-phosphate, 10 ng/ml platelet derived growth factor (PDGF), 10 ng/ml epithelial growth factor (EGF), 100 U/ml penicillin and 100 U/ml streptomycin. The stem cells (10×10⁶) were seeded and cultured in stem cell medium containing 2% fetal calf serum (FCS). After 12 hours, the culture was replaced with the labeling medium described above and incubated for an additional 24 hours.

### Example 4

### In vivo Protocol

The animal model preparation, catheter based coronary artery stem cell delivery, and physiological experiments using MRI/MRS were described in Example 1. To target tagged stem cells in vivo, first passage swine MSCs were cultured and transfected with Ad5-RSV-LacZ. The cells were tagged with annexin using an anti-CD44 antibody as described above in Example 3, which directs the stem cell toward the infarcted area by annexin and PS binding. After assessment of tagging efficiency, either intravenous or catheter based coronary artery administration of approximately 20×10⁶ cells/ml saline were infused and then flushed with 1 ml of saline. Sixteen days later, LV function and energetics were examined with MRI/MRS as described above in Example 2.

The LV was excised and the following experiments were performed to evaluate the fate of the tagged transplanted MSC: (a) gross specimen β-glactosidase staining to evaluate engraftment of cells by visible blue color; (b) histological sections with β-glactosidase staining to count cells expressing β-glactosidase under the light microscope as compared to MSC transplantation with no tags; (c) immunohistochemical staining using different antibodies to detect specific myocardial proteins (e.g., cardiac-specific troponin T) to identity cells derived from MSCs, and to look for gap junctions; and (d) polymerase chain reaction (PCR) of the frozen samples to amplify the Ad5-RSV-LacZ vector fragment DNA sequence to confirm that the β-glactosidase signals were from the transplanted cells and not from endogenous immune cells that can express low levels of β-glactosidase.

### Example 5

### In vitro Tagging of MSCs with Annexin

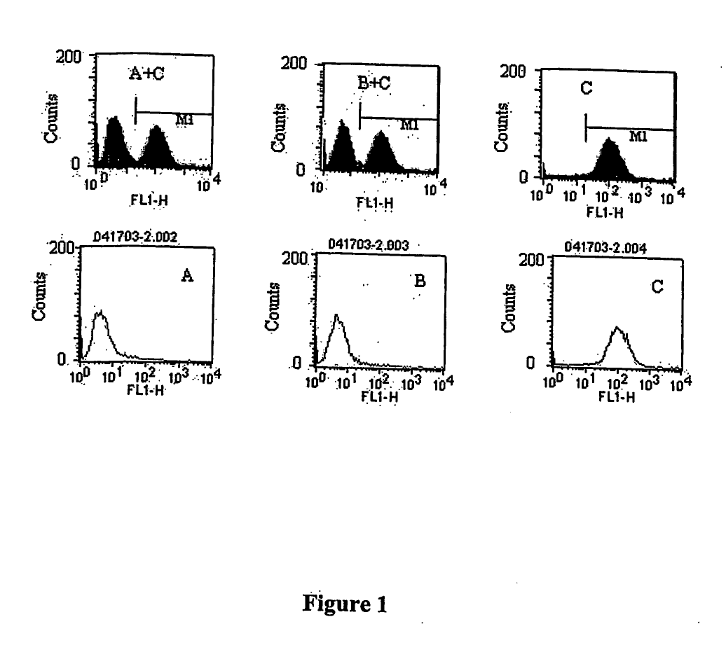

Data from in vitro studies demonstrated successful tagging of MSC with annexin, and showed that tagged MSC bind to apoptotic Jurkat cells (FIG. 1). Histograms of flow cytometry of MSCs with or without tagging are shown in FIG. 1. Panel A demonstrates that MSCs without tags interacted with FITC-anti-annexin antibody only. The fluorescence counts represent the FITC-IgG. Results indicated that MSCs do not have cell surface annexin. Panel B demonstrates that MSCs tagged with anti-CD44 antibody and crosslinked with annexin interacted with FITC-IgG. The fluorescence counts represent the FITC-IgG. This experiment was done as a negative control for the Panel C experiment. Panel C demonstrates that MSCs tagged with anti-CD44 antibody crosslinked with annexin interacted with FITC anti-annexin antibody. The fluorescence intensity appeared different as a consequence of binding to the stem cells. The top row shows the histograms from Panel A, B, and/or C combined as indicated. These data demonstrated that linking of annexin to MSC was greater than 90% since the two peaks had almost no overlap.

Immunohistochemistry also was used to demonstrate the specificity of annexin-tagged MSCs. Ad5-RSV-LacZ infected and annexin tagged MSCs (5×10⁵) were co-incubated with apoptotic Jurkat cells (5×10⁶) in cold binding buffer. For inducing apoptosis, Jurkat cells were pretreated with 0.5 μg/ml actinomycin D in 10% FBS-RPMI 1640 medium at 37° C. for 15 hrs. Cell smears were made for in situ Jurket cell death demonstration using TUNEL technology (In Situ Cell Death Detection Kit, Roche). The MSCs tagged with anti-CD44 and crosslinked to annexin bind and form a rosette with apoptotic cells surrounding the MSC cell. β-galactosidase expressed by MSCs was demonstrated using the X-Gal Staining Kit (Invitrogen).

### Example 6

### Annexin-Tagged MSCs Bind to Apoptotic Jurkat Cells

Ad5-RSV-LacZ transfected and annexin-tagged MSCs (5×10⁵) were co-incubated with apoptotic Jurkat cells (5×10⁶) in cold binding buffer for 2 hrs, which was then replaced with stem cell medium and the cells cultured at 37° C. for an additional 2 hrs. For inducing apoptosis, Jurkat cells were pretreated with 0.5 μg/ml actinomycin D in 10% FBS-RPMI 1640 medium at 37° C. for 15 hrs. Cell smears were made to demonstrate β-galactosidase expression in the transducted MSCs (X-Gal Staining kit, Invitrogen). MSCs bound several apoptotic Jurkat cells and began spreading along the substratum of the culture dish.

### Example 7

### In vivo Study of Tagged MSC Transplantation

Twenty million annexin-tagged allogenic MSCs were delivered through an ear vein catheter to two pigs prepared as described above in Example 1. Light microscopic evaluation indicated that MSCs homed in to the periscar region and were surviving and differentiating in the myocardial infarct region, which was not observed in two control animals in which untagged cells were delivered via a peripheral vein.

When the annexin tagged MSCs (20 million) were delivered via a LAD catheter in a separate in vivo study, significantly more stem cells homed to the myocardial infarct region. These results indicate that the annexin tagging system does promote stem cell homing into damaged myocardium, particularly when the cells are delivered via a coronary vein catheter.

### Example 8

### Site-Specific Directing and Non-Invasive Monitoring of Transplanted Stem Cells in vitro

To track transplanted stem cells' migration towards the target tissue or organ (e.g., myocardial infract (MI) and surrounding area), in vivo and in vitro transplanted MSCs were tagged with a novel triple-tag (a superparamagnetic nanoparticle and dual specific antibodies, wherein one antibody binding site is the stem cell surface antigen, CD44, and the other is annexin). Cells were labeled with superparamagnetic iron oxide particles (SPIO) by incubating non-labeled MSCs with SPIO, mixing for 30 min at 4° C., and washing 3 times with PBS.

Triple-tagged MSCs were resuspended in 100 ml of 1% low melt agarose at a cell density of 1×10⁷ cells/ml and loaded between two layers of agarose gel. The MRI detection was done using a 1.5 T magnet. A 2D gradient echo (GE) imaging technique with multiple slice interleave data acquisition scheme was applied. Data matrix: 256×256, TR/TE=600/30 msec; FOV=200 mm, slice thickness=3 mm. A small circularly polarized birdcage coil (12 cm ID) was used. This study demonstrated that MSCs labeled with an SPIO surface marker were clearly detectable in vitro with MRI, and therefore demonstrated the feasibility of site-specific targeting and non-invasive tracking of transplanted stem cells using MRI.

### Example 9

### Autologous MSCs Transplanted Through a Coronary Artery

To examine the proliferation and differentiation potential of autologous MSCs in vivo in the ischemic heart, experiments in 8 pigs with autologous MSC transplantation were performed. An LAD occlusion was performed to examine areas with cell transplantation and areas remote from the cell transplantation. Animals were followed for 2-3 weeks. An open chest MRS study was performed to examine the LV thickening fraction and myocardial energetics. Less scar thinning (no LV aneurysm formation) and akinesis were observed in the area where stem cells had been infused. The findings were consistent for all 7 pigs studied. On post-mortem examination, cells with β-gal staining were found in the infarcted area. The PCr/ATP high energy phosphate ratio was ˜1.0 in the area with cell transplantation. ³¹P-MRS were acquired using an ISIS column of 10×10 mm² perpendicular to the surface coil so that the phosphorous signal was from the area perfused by the occluded artery (where MSCs were seeded). This PCr/ATP ratio was compared to ˜0 in LV infarct without cell transplantation, ˜1.40 in failing hearts; and ˜2.2 in normal hearts. The finding of high-energy phosphates (PCr and ATP) present in areas where MSCs were transplanted indicates the presence of MSC engraftment.

These data indicate that: 1) the ischemic cardiac environment is permissive for stem cell differentiation; 2) high-energy phosphate metabolism is significantly different between injured myocardium with or without stem cell transplantation; and 3) autologous MSCs differentiate to myocytes in ischemic myocardium of swine hearts.

### Example 10

### Results

The use of MRI to dynamically track exogenously transplanted cells in vivo is an important advance in assessing treatments of genetic and degenerative diseases using cellular therapy. In vivo MRI observation, combined with histocytological analysis of excised tissue and retrieval of magnetically-labeled cells, results in a better understanding of engraftment and regeneration potential of transplanted cells. These studies also provide valuable data examining the potential of contrast-enhanced MRI to ultimately replace histological examination for cell therapy.

To visualize and track transplanted cells in vivo, the MSCs transduced with the Ad5-RSV-LacZ gene were labeled with a magnetic resonance contrast agent and the cells were tagged with a bi-specific antibody in which one of the component binding sites was directed against the stem cell surface antigen, CD44, while the other component binding site was directed against a target site (i.e., infarct region) antigen. Approximately 2×10⁷ cells/ml of saline was delivered into the pericardial space or directly injected into the ischemic region following acute myocardial infarction produced by ligation of the first diagonal coronary artery in pigs. MRI imaging was used to assess migration and location of the transplanted cells, as well as LV function and LV wall thickness, immediately after and at weekly intervals for 5 consecutive weeks to track the fate of the transplanted cells over an extended interval of time.

After the final MRI measurement, the heart was excised and examined to assess the effect of magnetic labeling of stem cells with bi-specific antibodies. For example, gross specimens were obtained to evaluate engraftment of LacZ-expressing cells based upon β-galactosidase staining. In addition, MRI examination of excised heart (especially scar and periscar regions) was performed to confirm the in vivo MRI results. Sections from excised heart tissue (e.g., scar and periscar regions) are stained for iron (Perls' Prussian blue reaction) and β-galactosidase (LacZ) expression, in combination with immunohistochemical staining (such as Troponin T), to assess and validate the different means of detecting and identifying the engrafted cells. Excised fresh heart tissue (periscar myocardium) was enzymatically dispersed and magnetically loaded cells were retrieved with a magnetic column to analyze and confirm their engraftment and cellular fate. The data obtained in the experiments described herein were compared with data from experiments using untagged MSCs.

The data from the in vitro studies demonstrated that tagging the MSCs with annexin was successful. Therefore, the present experiments demonstrated that tagged stem cells delivered through a peripheral vein can home into a myocardial infarct area. Homing to a mycoarcial infarct area did not occur in experiments using untagged stem cells delivered via the peripheral vein. The same strategy ws used with MSCs tagged via avidin/biotin with a MION antibody. These experiments allowed imaging (using, for example, MRI) of the location and trafficking of the MION-labeled cells, thereby facilitating evaluation of methods to enhance homing of the cells into the region of injured or infarcted myocardium. Based on the results herein, tagged and labeled autologous MSCs can be used clinically to increase MSC engraftment with a nonsurgical mode, and to follow cell trafficking non-invasively with MRI in cellular therapy for cardiac repair.

In another embodiment, the autologous MSCs were linked to complement (C3 or C5) using the same avidin/biotin binding system. The immunological response to complement deposition in areas of myocardial injury was known to cause further tissue damage. Binding of complement (C5, for example) to MSCs can direct the stem cells to find their “niches” in injured areas of the heart and compete with endogenous complement binding, thereby reducing complement deposition-induced cell injury.

### Example 11

### Direct Stem Cell Homing: Surface Modified MyoD−/− Cells

Although myoblasts may not be the optimal choice for stem cell therapy after myocardial infarction due to reported arrhythmias upon engraftment, MyoD−/− myoblasts have several characteristics that make them advantageous for this study. MyoD−/− myoblasts can be cultured in vitro for at least 30 passages, and they continuously express high levels of surface proteins through which a molecular bridge to Annexin V can be made. MyoD is expressed only in skeletal muscle and its precursors; it is repressed by specific genes in non-muscle cells. The removal of the MyoD gene allows the myoblasts to preserve their primitive state and prevents them from differentiating spontaneously into skeletal muscle. Since MyoD regulates skeletal muscle differentiation, knocking out MyoD may allow the myoblasts to differentiate into cardiomyocytes or endothelial cells upon injection into an infracted myocardium.

Annexin V was attached to the cell surface of MyoD−/− cells using the method described above in Example 3. Myocardial infarction and ischemia were produced by a ligation of the left coronary artery in mice. After the chest was closed, either 1×10⁶ or 2×10⁶ LacZ expressing MyoD−/− myoblasts were injected via the femoral vein. Mice underwent echocardiographic assessments and were sacrificed six days after induction of myocardial infarction and cell delivery. Whole heart X-gal staining revealed significant engraftment of Annexin V modified cells (n=5), while no “blue” cells were observed in the hearts of mice injected with unlabeled cells (n=3). Preliminary data strongly suggest an increase in homing and engraftment efficiency by MyoD−/− myoblasts tagged with Annexin V. The surface modified stem cells were delivered via peripheral vein route and were directed to a specific tissue in a given organ.

To quantify engraftment, hearts were cut into 8 μm sections and LacZ positive “blue” cells were counted. The differentiation fate of engrafted cells was determined by immunohistochemical and immunofluorescence staining of myogenic and endothelial markers.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of directing cells to a damaged or diseased tissue or organ in an individual, comprising the steps of:
providing a tagged cell, wherein said cells are tagged with a target cell binding member; and
introducing the tagged cell into the vasculature of said individual, thereby directing said cells to said damaged or diseased tissue or organ.

2. The method of claim 1, wherein said cells are stem cells.

3. The method of claim 2, wherein said stem cells are selected from the group consisting of mesenchymal stem cells (MSCs), and endothelial progenitor stem cells.

4. The method of claim 1, wherein said cells are autologous, allogeneic, or xenogeneic relative to said individual.

5. The method of claim 1, wherein said target cell binding member is selected from the group consisting of annexin, an antibody having specific binding affinity for cardiac-specific troponin T, an antibody having specific binding affinity for cardiac-specific troponin I, an antibody having specific binding affinity for skeletal muscle-specific troponin T, an antibody having specific binding affinity for skeletal muscle-specific troponin I, and an antibody having specific binding affinity for myosin.

6. The method of claim 1, wherein said introducing is via a coronary vein, a peripheral vein, or a coronary artery of said individual.

7. The method of claim 1, wherein-said damaged tissue or organ is selected from the group consisting of mycocardial, pericardial, pancreatic, kidney, skeletal muscle, central nervous system, and liver.

8. The method of claim 1, wherein said tagged cells further comprise an imaging agent.

9. The method of claim 8, wherein said imaging agent is selected from the group consisting of monocristalline iron oxide nanoparticle (MION), superparamagnetic iron oxide particles (SPIO), and ultra small superparamagnetic iron oxide (USPIO).

10. The method of claim 8, wherein said imaging agent is used for imaging said tagged cells.

11. A method of delivering stem cells to a myocardial infarction in an individual, comprising the steps of:
providing tagged stem cells, wherein said stem cells are tagged with annexin; and
introducing the tagged stem cell into the vasculature of said individual, thereby delivering said stem cells to said myocardial infarction.

12. The method of claim 11, wherein said stem cells are selected from the group consisting of MSCs and EPCs.

13. A composition comprising:
at least one linker moiety; and
at least one target cell binding member.

14. The composition of claim 13, wherein said target cell binding member is 30 selected from the group consisting of annexin, an antibody having specific binding affinity for cardiac-specific troponin T, an antibody having specific binding affinity for cardiac-specific troponin I, an antibody having specific binding affinity for skeletal muscle-specific troponin T, an antibody having specific binding affinity for skeletal muscle-specific troponin I, and an antibody having specific binding affinity for myosin.

15. The composition of claim 13, further comprising an imaging agent.

16. The composition of claim 15, wherein said imaging agent is selected from the group consisting of MION, SPIO, and USPIO.

17. An article of manufacture, comprising the composition of claim 13, and instructions for tagging cells with said target cell binding member using said linker, wherein said cells are stem cells harvested from an individual.

18. The article of manufacture of claim 17, further comprising instructions for performing an autologous transplant on said individual with said cells after said tagging.

19. Isolated stem cells, wherein said stem cells are tagged with a heterologous target cell binding member.

20. The stem cells of claim 19, wherein said stem cells are further labeled with an imaging agent.
